(19) United States
(12) Patent Application Publication
(10) Pub. No.: US 2021/0052564 A1
(43) Pub. Date: Feb. 25, 2021

(54) USE OF PANTOPRAZOLE IN PREVENTION AND TREATMENT OF NONALCOHOLIC FATTY LIVER DISEASE

(71) Applicant: Biguang TUO, Guizhou (CN)

(72) Inventors: Biguang Tuo, Guizhou (CN); Jiaxing Zhu, Guizhou (CN); Guorong Wen, Guizhou (CN); Xuemei Liu, Guizhou (CN); Rui Xie, Guizhou (CN); Xiaoying Long, Guizhou (CN); Hai Jin, Guizhou (CN); Jiaxing An, Guizhou (CN); Jingyu Xu, Guizhou (CN)

(21) Appl. No.: 16/983,955

(22) Filed: Aug. 3, 2020

(30) Foreign Application Priority Data
Aug. 19, 2019 (CN) 201910763474.4

(51) Int. Cl. A61K 31/4439; A61P 1/16

(52) U.S. Cl. CPC A61K 31/4439; A61P 1/16

(57) ABSTRACT

The present invention belongs to the technical field of new use of medicaments, and in particular relates to use of pantoprazole in the prevention and treatment of nonalcoholic fatty liver disease. The pantoprazole can effectively inhibit the expression of fatty acid-binding protein FABP1 in liver cells, suppress the occurrence of fatty liver disease caused by a high-fat diet, reduce the deposition of fat in liver, and decrease the content of triglycerides in liver tissue, thereby achieving the purpose of preventing and treating nonalcoholic fatty liver disease.

### CROSS-REFERENCE TO RELATED APPLICATIONS

The present application claims priority to and the benefit of Chinese Patent Application Number 201910763474.4 filed on (or having a priority date of) Aug. 19, 2019 and entitled “USE OF PANTOPRAZOLE IN PREVENTION AND TREATMENT OF NONALCOHOLIC FATTY LIVER DISEASE”, which is incorporated by reference herein in its entirety for all intents and purposes.

### TECHNICAL FIELD

The present invention belongs to the technical field of new use of medicaments, and particularly relates to use of proton pump inhibitor pantoprazole (PPZ) in the prevention and treatment of nonalcoholic fatty liver disease.

### BACKGROUND

Nonalcoholic fatty liver disease refers to a clinical pathological syndrome not caused by alcohol or other clear liver-damaging factors, characterized by the degeneration and accumulation of fat in hepatocytes. Over time, nonalcoholic fatty liver disease will further cause steatohepatitis, liver fibrosis, cirrhosis and liver cancer. In addition to directly causing cirrhosis and hepatocellular carcinoma, nonalcoholic fatty liver disease can also affect the development of other chronic liver diseases and play a role in the onset of type 2 diabetes and atherosclerosis. Therefore, the active prevention and treatment of fatty liver disease is very important to prevent the progression of chronic liver disease and improve the prognosis.

At present, nonalcoholic fatty liver disease is mainly treated clinically by blood lipid decrease, blood glucose reduction, liver protectant administration and lifestyle change such as weight loss and exercise. Clinically, there are no effective medicaments for treating nonalcoholic fatty liver disease.

Nonalcoholic fatty liver disease is caused by excessive accumulation of lipids (mainly in the form of triglycerides) in the liver, with a main characteristic of diffuse hepatocellular bullous steatosis.

PPZ, a proton pump inhibitor, can effectively inhibit H+-K+-ATPase in parietal cells. At present, PPZ has been widely used in the clinical treatment of gastric acid-related diseases (such as peptic ulcer disease and gastroesophageal reflux disease).

However, so far, use of proton pump inhibitor PPZ has not been found in the prevention and treatment of nonalcoholic fatty liver disease.

### SUMMARY

In view of this, the present invention is intended to provide use of proton pump inhibitor PPZ in the prevention and treatment of nonalcoholic fatty liver disease, and thus provide new use of pantoprazole.

In order to realize the objective of the present invention, the present invention provides the following technical solutions.

The present invention provides use of pantoprazole in the prevention and treatment of nonalcoholic fatty liver disease.

Preferably, the pantoprazole prevents and treats nonalcoholic fatty liver disease by inhibiting the expression of fatty acid-binding protein FABP1 in liver cells.

Preferably, the pantoprazole inhibits nonalcoholic fatty liver disease caused by a fatty diet.

Preferably, the pantoprazole prevents and treats nonalcoholic fatty liver disease by reducing the deposition of fat in liver.

Preferably, the pantoprazole prevents and treats nonalcoholic fatty liver disease by reducing the content of triglycerides in liver tissue.

The present invention also provides use of a composition including pantoprazole in the prevention and treatment of nonalcoholic fatty liver disease.

Preferably, the composition includes pantoprazole as the only active substance.

The present invention provides use of pantoprazole in the prevention and treatment of nonalcoholic fatty liver disease. The pantoprazole can effectively inhibit the expression of fatty acid-binding protein FABP1 in liver cells, suppress the occurrence of fatty liver disease caused by a high-fat diet, reduce the deposition of fat in liver, and decrease the content of triglycerides in liver tissue, thereby achieving the purpose of preventing and treating nonalcoholic fatty liver disease.

### BRIEF DESCRIPTION OF DRAWINGS

FIG. 1 shows the effect of PPZ (proton pump inhibitor) on the mRNA and protein expression for fatty acid-binding protein FABP1 in human normal liver cell lines LO2 and QSG-7701 and hepatocellular carcinoma cell line Huh7, where, different dosages of PPZ are added to act on liver cells for 24 h, and the mRNA and protein expression for fatty acid-binding protein FABP1 in liver cells is detected by RT-PCR and Western Blot; and the results show that PPZ inhibits the mRNA and protein expression for fatty acid-binding protein FABP1 in human liver cells in a dosage-dependent manner, with n=8 in each series, and as compared with the control group, *P<0.05 and **P<0.01.

FIG. 2 shows the effect of PPZ on liver weight in mice given a high-fat diet, where, compared with the normal group (not given a high-fat diet), the group given a high-fat diet has a significantly-increased liver weight; compared with the control group, mice in each dosage group of PPZ have a significantly-reduced liver weight, exhibiting no dosage-dependence; as compared with the normal group, $P<0.05; and as compared with the control group, *P<0.05.

FIG. 3 shows the effect of PPZ (proton pump inhibitor) on hepatic steatosis in mice caused by a high-fat diet, where the liver tissue is embedded in paraffin and sectioned, and hepatic steatosis is evaluated by liver histology HE staining; compared with the normal group (not given a high-fat diet), the group given a high-fat diet has significant steatosis in liver; compared with the control group, mice of each dosage group of PPZ have significantly-alleviated hepatic steatosis, exhibiting dosage-dependence; the upper part shows representative histological change images for all groups; the lower part shows the comparison of steatosis degrees for all groups; as compared with the normal group, $$P<0.05; and as compared with the control group, *P<0.05 and **P<0.01.

FIG. 4 shows the effect of PPZ (proton pump inhibitor) on the fat deposition in liver of a mouse given a high-fat diet, where, the liver tissue is frozen and sectioned, and the fat deposition in liver is assayed by Oil Red O (ORO) staining; compared with the normal group (not given a high-fat diet), the group given a high-fat diet has significantly-increased fat deposition in liver; compared with the control group, mice of each dosage group of PPZ have significantly-alleviated fat deposition in liver, exhibiting dosage-dependence; as compared with the normal group, $$P<0.05; and as compared with the control group, *P<0.05 and **P<0.01.

FIG. 5 shows the effect of PPZ (proton pump inhibitor) on the triglyceride content in liver tissue of a mouse given a high-fat diet, where, 100 mg of mouse liver tissue is collected and homogenized, and the content of triglyceride is detected by a biochemical method; the result is expressed by the amount (mg) of triglyceride per gram of liver tissue (mg/g of liver); compared with the normal group (not given a high-fat diet), the group given a high-fat diet has a significantly-increased content of triglyceride in liver tissue; compared with the control group, mice of each dosage group of PPZ have a significantly-reduced content of triglyceride in liver tissue, exhibiting dosage-dependence; as compared with the normal group, $$P<0.05; and as compared with the control group, *P<0.05 and **P<0.01 (TG: triglyceride).

### DETAILED DESCRIPTION

The present invention provides use of pantoprazole in the prevention and treatment of nonalcoholic fatty liver disease. The pantoprazole prevents and treats nonalcoholic fatty liver disease by inhibiting the expression of fatty acid-binding protein FABP1 in liver cells. In the present invention, the pantoprazole inhibits nonalcoholic fatty liver disease caused by a fatty diet. In the present invention, the pantoprazole prevents and treats nonalcoholic fatty liver disease by reducing the deposition of fat in liver. In the present invention, the pantoprazole prevents and treats nonalcoholic fatty liver disease by reducing the content of triglycerides in liver tissue.

The present invention also provides use of a composition including pantoprazole in the prevention and treatment of nonalcoholic fatty liver disease. In the present invention, the composition preferably includes pantoprazole as the only active substance.

The technical solutions of the present invention are described below in connection with specific examples, but the claimed scope of the present invention is not limited thereto.

### Example 1

Effect of PPZ (Proton Pump Inhibitor) on the Fatty Acid-Binding Protein in Human Liver Cells

Fatty acid-binding proteins play an important role in the uptake of fatty matters by liver cells. Normal liver cell lines LO2 and QSG-7701 and hepatocellular carcinoma cell line Huh7 were cultivated in vitro for 48 h using medium. Then 20 uM, 40 uM, 80 uM, 160 uM, and 320 uM PPZ (PPZ, proton pump inhibitor) solutions and a control solution (saline) were added to the medium separately, and acted on cells for 24 h in vitro. The cells were collected, and the mRNA and protein expression for fatty acid-binding protein FABP1 was analyzed by PCR and Western Blot in these cells treated with different dosages of PPZ.

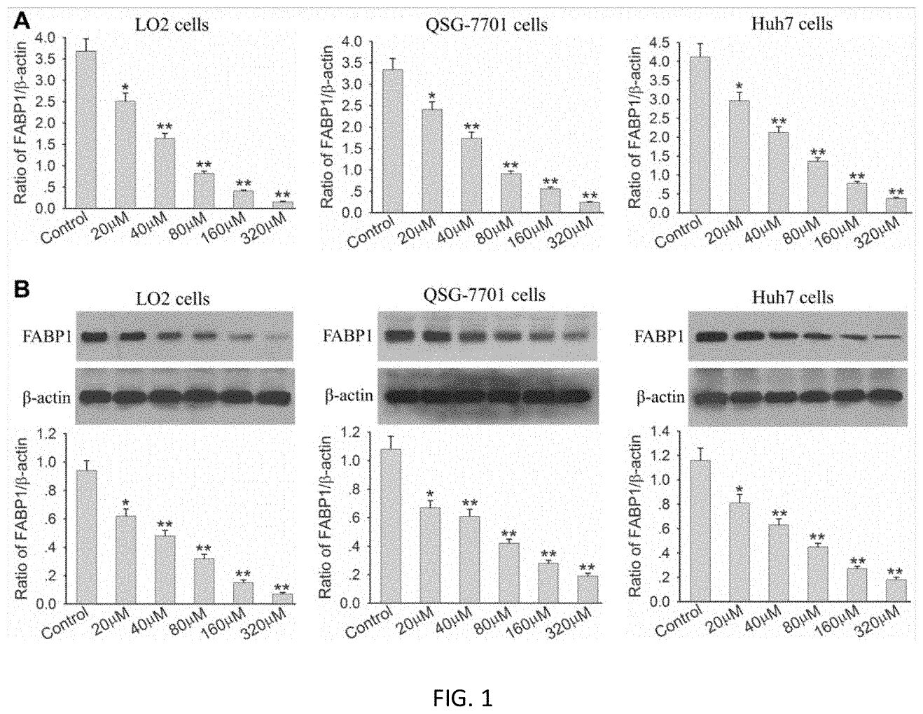

The results showed that PPZ inhibited the mRNA and protein expression of fatty acid-binding protein FABP1 in human liver cells in a dosage-dependent manner (see FIG. 1), with n=8 in each series. As compared with the control group, *p<0.05, **P<0.01.

### Example 2

Effect of PPZ (Proton Pump Inhibitor) on the Occurrence of Fatty Liver Disease and the Deposition of Fat in Liver Caused by a High-Fat Diet in Mice

Mice with fatty liver disease caused by a high-fat diet are animal models of nonalcoholic fatty liver disease commonly used in experimental research. In order to study the effect of PPZ (proton pump inhibitor) on the occurrence of nonalcoholic fatty liver disease, 5 to 6 week-old male C57BL6 mice were adopted for experiment. The mice were given a high-fat diet to establish nonalcoholic fatty liver disease models, and then the effects of different dosages of PPZ on the occurrence of fatty liver disease in mice were observed. After the mice had been given a high-fat diet for four weeks, the mice were randomly divided into a control group and PPZ (proton pump inhibitor) groups of 1 mg/kg, 2 mg/kg, 5 mg/kg and 10 mg/kg, with each group of 12 mice. PPZ, at dosages of 1 mg/kg, 2 mg/kg, 5 mg/kg and 10 mg/kg, or control solution (saline) were intraperitoneally administered to mice once every 12 hours. 10 weeks after administration, mice were sacrificed, and the occurrence of fatty liver disease and the deposition of fat in liver were examined in mice. 100 mg of mouse liver tissue was collected and homogenized. The content of triglyceride was detected by a biochemical method, and the result was expressed by the amount (mg) of triglyceride per gram of liver tissue (mg/g of liver).

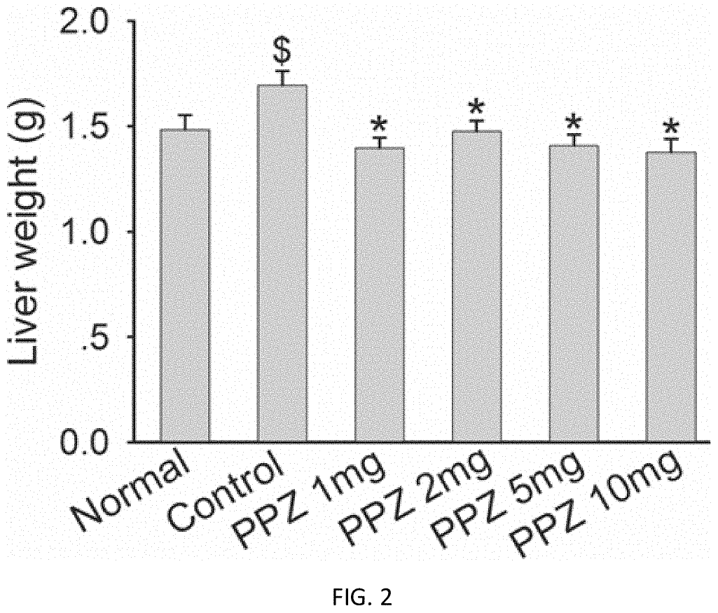

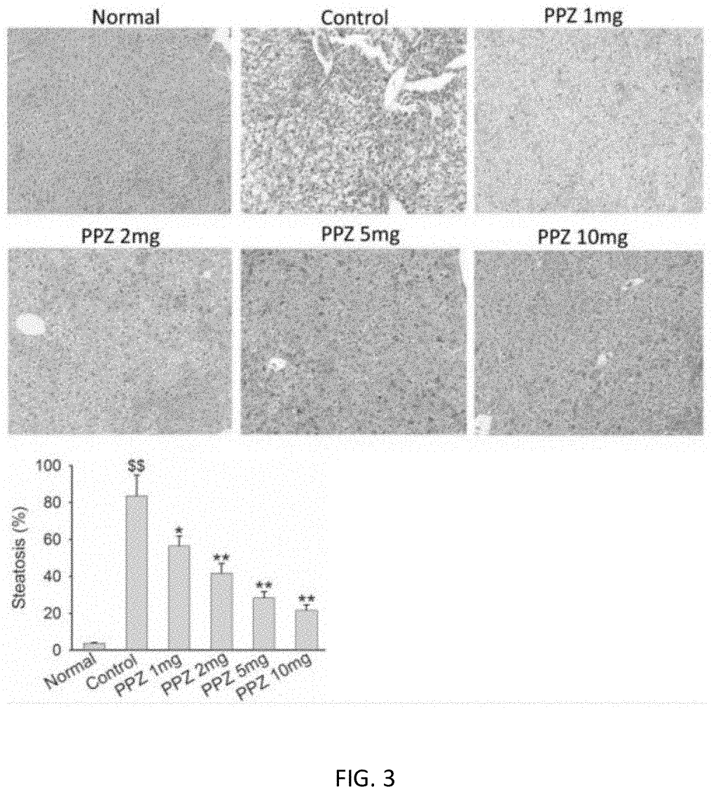

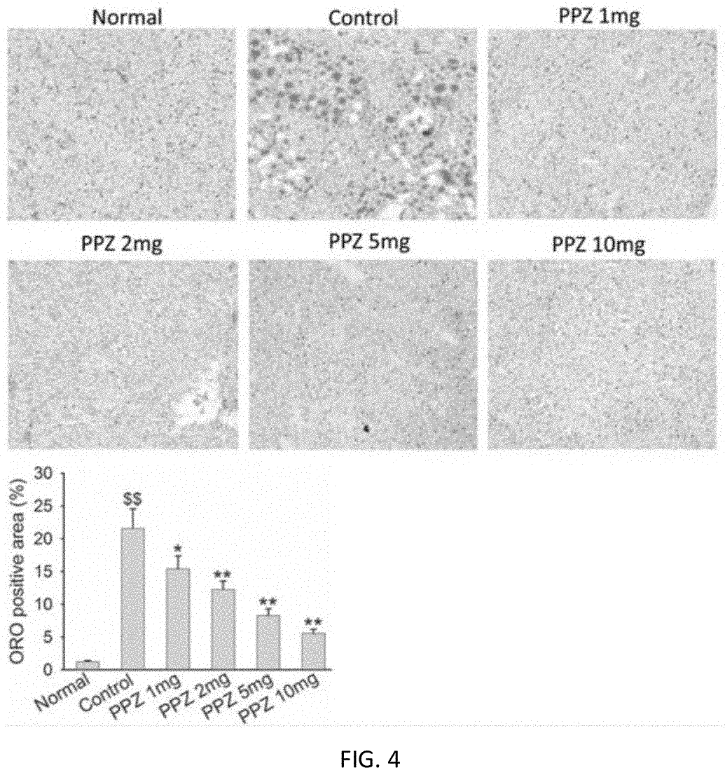

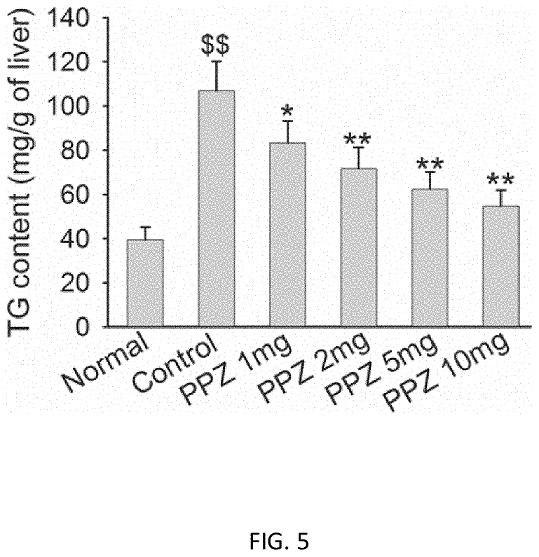

As shown in FIG. 2, compared with the normal group (not given a high-fat diet), the group given a high-fat diet has a significantly-increased liver weight; compared with the control group, mice in each dosage group of PPZ have a significantly-reduced liver weight, exhibiting no dosage-dependence; as compared with the normal group, $P<0.05; and as compared with the control group, *P<0.05. As shown in FIG. 3, compared with the normal group (not given a high-fat diet), the group given a high-fat diet has significant steatosis in liver; compared with the control group, mice of each dosage group of PPZ have significantly-alleviated hepatic steatosis, exhibiting dosage-dependence; the upper part shows representative histological change images for all groups; the lower part shows the comparison of steatosis degrees for all groups; as compared with the normal group, $$P<0.05; and as compared with the control group, *P<0.05 and **P<0.01. As shown in FIG. 4, compared with the normal group (not given a high-fat diet), the group given a high-fat diet has significantly-increased fat deposition in liver; compared with the control group, mice of each dosage group of PPZ have significantly-alleviated fat deposition in liver, exhibiting dosage-dependence; as compared with the normal group, $$P<0.05; and as compared with the control group, *P<0.05 and **P<0.01. As shown in FIG. 5, compared with the normal group (not given a high-fat diet), the group given a high-fat diet has a significantly-increased content of triglyceride in liver tissue; compared with the control group, mice of each dosage group of PPZ have a significantly-reduced content of triglyceride in liver tissue, exhibiting dosage-dependence; as compared with the normal group, $$P<0.05; and as compared with the control group, *P<0.05 and **P<0.01 (TG: triglyceride).

These results indicate that PPZ (proton pump inhibitor) can significantly inhibit the expression of fatty acid-binding protein in liver cells, effectively suppress the occurrence of fatty liver disease in mice caused by a high-fat diet, reduce the deposition of fat in liver of a mouse and decrease the content of triglyceride in liver tissue. It is feasible to prepare a medicament that can effectively prevent and treat nonalcoholic fatty liver disease with PPZ (proton pump inhibitor).

The foregoing descriptions are only preferred implementation manners of the present invention. It should be noted that for a person of ordinary skill in the art, several improvements and modifications may further be made without departing from the principle of the present invention. These improvements and modifications should also be deemed as falling within the protection scope of the present invention.

What is claimed is:

1. A method comprising the use of pantoprazole in the prevention and treatment of nonalcoholic fatty liver disease.

2. The method of claim 1, wherein the pantoprazole prevents and treats nonalcoholic fatty liver disease by inhibiting the expression of fatty acid-binding protein FABP1 in liver cells.

3. The method of claim 1, wherein the pantoprazole inhibits nonalcoholic fatty liver disease caused by a fat diet.

4. The method of claim 1, wherein the pantoprazole prevents and treats nonalcoholic fatty liver disease by reducing the deposition of fat in liver.

5. The method of claim 1, wherein the pantoprazole prevents and treats nonalcoholic fatty liver disease by reducing the content of triglycerides in liver tissue.

6. A method comprising the use of a composition comprising pantoprazole in the prevention and treatment of nonalcoholic fatty liver disease.

7. The method of claim 6, wherein the composition comprises pantoprazole as the only active substance.
